(19) United States
(12) Patent Application Publication
(10) Pub. No.: US 2024/0293222 A1
(43) Pub. Date: Sep. 5, 2024

(54) NIPPLE RECONSTRUCTION IMPLANT

(71) Applicant: Tepha, Inc., Lexington, MA (US)

(72) Inventors: Skander Limem, Lynnfield, MA (US); Kemal Sariibrahimoglu, Sunnyvale, CA (US); Jeffrey Robert Scott, Foxborough, MA (US); Simon F. Williams, Cambridge, MA (US); Timothy John Butler, Pelham, NH (US); German Oswaldo Hohl Lopez, East Boston, MA (US)

(73) Assignee: Tepha, Inc., Lexington, MA (US)

(21) Appl. No.: 18/559,832

(22) PCT Filed: May 9, 2022
(86) PCT No.: PCT/US2022/028284

§ 371 Date: Nov. 9, 2023

Related U.S. Application Data
(60) Provisional application No. 63/187,010, filed on May 11, 2021.

(51) Int. Cl. A61F 2/12; A61F 2/00; A61L 27/18; A61L 27/56; A61L 27/58

(52) U.S. Cl. CPC A61F 2/12; A61L 27/18; A61L 27/56; A61L 27/58; A61F 2002/0081; A61F 2210/0004; A61F 2210/0057; A61F 2230/0004; A61F 2230/005; A61F 2230/0069; A61F 2230/0093; A61L 2430/04

(57) ABSTRACT

Absorbable implants can be used to reconstruct the nipple with regenerated tissue resulting in improved aesthetic satisfaction. The implants are particularly suitable for use in plastic surgery procedures, for example, to reconstruct the nipple following total mastectomy and breast reconstruction. The implants may be formed from absorbable mesh and/or dry spun sheet.

### RELATED APPLICATIONS

Foreign priority benefits are claimed under 35 U.S.C. § 119(a)-(d) or 35 U.S.C. § 365(b) of U.S. application No. 63/187,010, filed May 11, 2021.

### FIELD OF THE INVENTION

The present invention generally relates to surgical implants, and more particularly, to three-dimensional porous implants suitable for reconstruction of a nipple.

### BACKGROUND OF THE INVENTION

Nipple reconstruction following certain mastectomy procedures has become an important component of breast cancer treatment for some patients because the surgery can provide the patient aesthetic and psychosocial benefits.

Several options for re-creating the appearance of the nipple on the breast are available. These options include prosthetic nipples, made for example from a silicone-based material, that may be temporarily secured to the patient's skin. These prostheses are however external devices attached with a temporary adhesive that will wear out over time, and are perceived as artificial.

Alternatively, a nipple may be reconstructed surgically using the patient's own tissue, or it may be reconstructed using an implant.

Surgically created nipples are permanent and have a more natural feel, but typically require donor skin and a second surgery to harvest suitable tissue. They also require the surgeon to construct a replacement nipple with the proper size, projection and shape which can be challenging when it needs to match the contralateral nipple.

Several nipple reconstruction implants have been developed to avoid the need to harvest suitable tissue for nipple reconstruction from a patient.

US20210052774 to Edwards discloses nipple reconstruction implants derived from acellular tissue matrices and three-dimensional biologic scaffolds.

WO2020081806 to Spector discloses surgical implants for nipple reconstruction comprising minced or zested cartilage that is encaged by an external biocompatible scaffold.

WO2020230997 to Choi discloses an implant for reconstruction of the nipple areolar complex (NAC) comprising a two-wheeled composite with a columnar body and a main body portion.

US2013/0211519 to Dempsey discloses a remodelable implant comprising a remodelable extracellular matrix material, such as an extracellular matrix sheet isolated in sheet form from a mammalian or other tissue source, and configured by rolling and/or molding, to provide a shaped implant.

US2016/0243286 to Collins discloses tissue engineered constructs for nipple reconstruction comprising cells, scaffolding and optionally other materials, such as nutrients and growth factors.

Notwithstanding the above, there is still a need for improved nipple reconstruction implants that, when implanted, can generate new tissue with a specific and desirable appearance and feel.

### SUMMARY OF THE INVENTION

Nipple implants described herein assist the surgeon in reconstructing the nipple-areola complex (NAC) following total mastectomy and breast reconstruction, enhancing the appearance of the breast, reconstructing lost or missing tissue, enhancing the tissue structure of NAC, restoring the natural feeling of soft tissue of the NAC, and delivering biological and synthetic materials to assist in tissue regeneration, repair, and reconstruction of the NAC.

In embodiments, the nipple implants are porous, providing a macroporous network for tissue ingrowth, and may further comprise collagen, cells, and fat. Following implantation, the implant is designed to be invaded by connective tissue, and become well integrated. In embodiments, the nipple implant comprises a cylindrical shape with first and second circular bases of the same circumference at each end of the cylindrical shape.

In embodiments, the implant further comprises a hemispherical shape, or dome shape, connected to the second circular base of the cylindrical shape of the implant.

In embodiments, the implant comprises a shell at least partly surrounding a macroporous network, and the shell comprises a cylindrical shape with first and second circular bases at each end of the cylindrical shape, and a hemispherical shape connected to the second circular base of the cylindrical shape.

In embodiments, the implant has a longitudinal axis with a height h measured longitudinally between a first end of the implant at one end of the axis and a second end of the implant at the opposite end of the axis.

In embodiments, the shell is porous.

In embodiments, the shell does not enclose the macroporous network at the first end of the implant.

In embodiments, the implant further comprises a flange. The flange is located at the first end of the implant. The flange has a larger circumference than the cylindrical shape of the implant so that the flange protrudes from the circular base of the cylindrical shape. In embodiments, the flange is porous. In embodiments, the flange is absorbable. The flange is designed to be placed on the breast mound and posterior to the second end of the implant when the implant with a flange is implanted in a patient.

In embodiments, the nipple implants comprise a load bearing macroporous network with an open pore structure.

In embodiments, the macroporous network of the implant is shaped to fill the shell of the implant. In embodiments, the macroporous network has a cylindrical shape connected to a hemispherical shape at one end.

In embodiments, the pores of the macroporous network have an average diameter or average width of 75 microns to 10 mm, and more preferably 100 microns to 2 mm.

In embodiments, the filaments of the implant have one or more of the following properties: an average diameter or average width of 10 microns to 5 mm, a breaking load of 0.1 to 200 N, an elongation to break of 10 to 1,000%, and an elastic modulus of 0.05 to 1,000 MPa.

In embodiments, filaments of the implants are formed with surface roughness (Ra). Surface roughness promotes cell attachment and tissue formation on the implants. Surface roughness also promotes attachment of the implant to neighboring tissues, encourages tissue ingrowth, and helps to prevent movement of the device after implantation. In embodiments, the implant comprises filaments having a surface roughness of 0.02 to 75 microns, more preferably 0.1 to 50 or 0.5 to 30 microns, and even more preferably 5 to 30 microns.

In embodiments, the implant has a shape and size suitable for use in nipple reconstruction. In embodiments, the height h of the implant is 0.1 to 2 cm, more preferably 0.5 to 1.5 cm, and even more preferably 0.3 to 1 cm. In embodiments, the diameter of the cylindrical shape of the implant is from 2 to 10 mm, and more preferably 4 to 7 mm.

In embodiments, the macroporous network comprises an absorbable polymer. In embodiments, the absorbable polymer has one or more of the following properties: (i) an elongation at break greater than 100%; (ii) an elongation at break greater than 200%; (iii) a melting temperature of 60° C. or higher; (iv) a melting temperature higher than 100° C.; (v) a glass transition temperature of less than 0° C.; (vi) a glass transition temperature between −55° C. and 0° C.; (vii) a tensile modulus less than 300 MPa; and (viii) a tensile strength higher than 25 MPa. In embodiments, the absorbable polymer comprises, or is prepared from, one or more monomers selected from the group: glycolide, lactide, glycolic acid, lactic acid, 1,4-dioxanone, trimethylene carbonate, 3-hydroxybutyric acid, 3-hydroxybutyrate, 3-hydroxyhexanoate, 4-hydroxybutyric acid, 4-hydroxybutyrate, 3-hydroxyoctanoate, ε-caprolactone, 1,4-butanediol, 1,3-propane diol, ethylene glycol, glutaric acid, malic acid, malonic acid, oxalic acid, succinic aid, or adipic acid, or the absorbable polymer comprises poly-4-hydroxybutyrate (P4HB) or copolymer thereof, or poly(butylene succinate) (PBS) or copolymer thereof. In embodiments, the implant comprises P4HB and copolymers thereof, or PBS and copolymers thereof, and is not crosslinked. In embodiments, the PBS polymer and copolymers may further comprise one or more of the following: branching agent, cross-linking agent, chain extender agent, and reactive blending agent. The PBS and P4HB polymers and copolymers may be isotopically enriched. In embodiments, the polymers used to prepare the implants have weight average molecular weights of 50 to 1,000 kDa, more preferably 90 to 600 kDa, and even more preferably from 200 to 450 kDa relative to polystyrene determined by GPC.

In embodiments, the implant is absorbable. The implants preferably comprise a polymeric material with a predictable rate of degradation, and a predictable strength retention in vivo. When the implants are absorbable, degradation of the implant can allow further invasion of the implant with tissue, and this process can continue until the implant is completely absorbed.

In embodiments, the implant further comprises one or more of the following: autologous fat, fat lipoaspirate, injectable fat, adipose cells, fibroblast cells, stem cells, gels, hydrogels, hyaluronic acid, collagen, antimicrobial agent, antibiotic agent, and bioactive agent.

In embodiments, the implants have anisotropic properties meaning that the implants have different properties in different directions.

In embodiments, the implant is shell-less, and optionally the perimeter edges of the implant are treated, for example, to remove barbs and make the implant generally smoother. The edges may be treated by, for example, trimming or heat treating.

In embodiments, the implant retains strength long enough to allow new tissue to fill the space occupied by the implant, and thereby maintain the shape of the nipple after implantation of the implant. The implant directs re-modeling of the patient's tissue to form the nipple. The implant preferably provides support for the nipple during this transition period. The shape of the nipple implant is maintained for a prolonged period in order to direct tissue ingrowth into the implant, and produce the desired nipple shape.

In embodiments, the macroporous network of the implant is at least partly filled with a degradable polymer. The degradable polymer is preferably degraded faster than the macroporous network. In embodiments, the macroporous network comprises a hydrogel.

In embodiments, the implant comprises macroporous absorbable mesh and a microporous dry spun sheet. In embodiments, the implant is formed by rolling a macroporous absorbable mesh into a cylinder to form the core of the implant, and wrapping the core with a microporous dry spun sheet. In embodiments, the macroporous absorbable mesh is a knitted monofilament mesh. In embodiments, the diameter of the monofilament is a size 5/0 or a size 6/0. In embodiments, the mesh and dry spun sheet are formed from poly-4-hydroxybutyrate or copolymer thereof.

In embodiments, the implant comprises an absorbable dry spun sheet rolled to form a cylindrical core of the implant, and a macroporous mesh wrapped around the core of the implant. In embodiments, the macroporous mesh is a knitted monofilament mesh. In embodiments, the diameter of the monofilament is a size 5/0 or a size 6/0. In embodiments, the mesh and dry spun sheet are formed from poly-4-hydroxybutyrate or copolymer thereof.

In embodiments, the implant comprises a macroporous absorbable mesh and a dry spun sheet folded to form a cylindrical shape with a flange, wherein the absorbable mesh is located in the core of the cylindrical shape and the dry spun is located on the outer surface of the implant. In embodiments, the macroporous mesh is a knitted monofilament mesh. In embodiments, the diameter of the monofilament is a size 5/0 or a size 6/0. In embodiments, the mesh and dry spun sheet are formed from poly-4-hydroxybutyrate or copolymer thereof.

In embodiments, the implants have an endotoxin content of less than 20 endotoxin units per implant.

In embodiments, the implants are sterilized implants. The implants can be sterilized by a range of techniques including without limitation ethylene oxide, electron beam, or gamma-irradiation.

In embodiments, the method of manufacturing the nipple implant further comprises enclosing the macroporous network at least partly in a shell by coating the macroporous network with a polymeric composition.

In embodiments, the implant includes a first portion of a polymeric knitted or woven macroporous textile and a second portion of polymeric microporous non-woven or foam, wherein the first portion and the second portion are configured to form a cylindrical body portion and at least a partially dome-shape at an end of the cylindrical body portion.

In embodiments, the implant includes a first portion of knitted or woven microporous poly-4-hydroxybutyrate or copolymer thereof and a second portion of spun poly-4-hydroxybutyrate or copolymer thereof. The first portion and second portion are configured to form a cylindrical body portion and at least a partially dome-shape at an end of the cylindrical body portion. In some embodiments, the first portion may be a core of the implant and the second portion surrounds the core. In some embodiments, a flange base is provided at an end of the implant opposite of the at least partially dome-shape end.

In embodiments, the implant includes an exterior body having a base, a hollow cylindrical portion projecting from the base, and at least a partially dome-shape at an end of the hollow cylindrical portion opposite the base. The exterior body defines an internal cavity and an interior load bearing body is located within and at least partially fills the internal cavity. Each of the exterior body and the interior load bearing body are formed of at least one of a knitted, woven or spun absorbable textile. In embodiments, the absorbable textile is formed from poly-4-hydroxybutyrate or copolymer thereof.

In embodiments, the implant includes an exterior body having a base, a hollow cylindrical portion projecting from the base, and at least a partially dome-shape at an end of the hollow cylindrical portion opposite the base. A load bearing body includes a base and a resilient structure projecting upwardly from the base. The exterior body defines an internal cavity and the resilient structure projects into the internal cavity. In some embodiments, the resilient structure is located inside the cylindrical portion and the at least partially dome-shape. In some embodiments, there are one or more gaps in the resilient structure when viewed in a radial direction. In some embodiments, the resilient structure has a polygon shape which may include a clover-leaf shape. In other embodiments, the resilient structure includes two or more adjacent windings of sheet material, where adjacent windings may have a gap therebetween or may be contiguous. The resilient structure may have a shape that is the same as or is different from the shape of the internal cavity. In some embodiments, the resilient structure has a corrugated surface.

In embodiments, the implant comprises a macroporous absorbable mesh folded to form a cone shape. In embodiments, the macroporous absorbable mesh is a monofilament mesh. In embodiments, a two-dimensional macroporous absorbable mesh is folded to form a three-dimensional macroporous mesh nipple implant. In embodiments, a two-dimensional triangular macroporous absorbable mesh is folded into a cone shape, and the shape fixated, for example, by heat scaling, stitching or gluing.

In embodiments, the methods of manufacturing the implants comprise adding one or more of the following components: autologous fat, fat lipoaspirate, injectable fat, adipose cells, fibroblast cells, stem cells, gel, hydrogel, hyaluronic acid, collagen, antimicrobial, antibiotic, and bioactive agent. In embodiments, these components are added to the macroporous network by coating, spraying, immersion or injection.

In embodiments, the implant is implanted by a method comprising: making an incision in a patient to create a tissue enclosure that is configured to receive a nipple implant; and inserting the nipple implant into the tissue enclosure, wherein the tissue enclosure is configured to conform around the nipple implant. In embodiments, the method of implanting the implant comprises configuring an incision to create tissue flaps with opposable edges, such that when the edges are brought together the tissue flaps form a void for receiving the nipple implant so that the inner surface of the tissue flaps are in contact with the nipple implant. In embodiments, the method of implanting the implant comprises making an incision with a CV-flap incision path, a S-flap incision path, or a star-flap incision path. In embodiments, the implant comprises a flange protruding from the cylindrical shape of the implant, and the implant is implanted in the patient with the flange positioned on the breast mound of the patient and posterior to the second end of the cylindrical shape. In embodiments, the implant comprises a hemispherical shape, and the implant is implanted so that the hemispherical shape is adjacent to the skin of the patient and anterior to the remainder of the implant.

In embodiments, the implant serves to provide the surgeon with a means to deliver cells, stem cells, differentiated cells, fat cells, muscle cells, platelets, tissues, lipoaspirate, extracellular adipose matrix proteins, gels, hydrogels, hyaluronic acid, collagen, bioactive agents, drugs, antibiotics, and other materials to the implant site.

In embodiments, the implants can be implanted to replace and or increase a soft tissue volume or a tissue mass.

These advantages as well as other objects and advantages of the present invention will become apparent from the detailed description to follow, together with the accompanying drawings.

### BRIEF DESCRIPTION OF THE DRAWINGS

FIG. 1A is a picture of a nipple implant formed with a core of macroporous P4HB monofilament knitted mesh and a microporous P4HB dry spun shell.

FIG. 1B is a picture showing an alternate view of the nipple implant shown in FIG. 1A.

FIG. 2 is a picture of a nipple implant formed from a composite of a microporous dry spun sheet and a macroporous absorbable mesh rolled into a cylinder.

FIG. 3 is a picture of a nipple implant formed from a 2D heat set sheet of monofilament P4HB mesh folded to form a 3D cone shape.

FIGS. 4A-E are pictures of a thermo-formed mesh nipple implant.

FIG. 5 is a picture of a loosely rolled mesh inner body and an outer mesh shell.

FIG. 6 is a picture of a relatively tightly rolled mesh nipple implant.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail).

Reference to a singular item, includes the possibility that there are plural of the same items present. More specifically, as used herein and in the appended claims, the singular forms “a.” “an,” “said” and “the” include plural referents unless the context clearly dictates otherwise. It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation.

To further assist in understanding the following definitions are set forth below. However, it is also to be appreciated that unless defined otherwise as described herein, all technical and scientific terms used herein have the same meaning as commonly understood by one of ordinary skill in the art to which this invention belongs.

### I. Definitions

“Absorbable” as generally used herein means the material is degraded in the body, and the degradation products are eliminated or excreted from the body. The terms “absorbable”, “resorbable”, “degradable”, and “erodible”, with or without the prefix “bio”, can be used interchangeably herein, to describe materials broken down and gradually absorbed, excreted, or eliminated by the body, whether degradation is due mainly to hydrolysis or mediated by metabolic processes.

“Bioactive agent” as generally used herein refers to therapeutic, prophylactic or diagnostic agents, preferably agents that promote healing and the regeneration of host tissue, and also therapeutic agents that prevent, inhibit or eliminate infection. “Agent” includes a single such agent and is also intended to include a plurality.

“Biocompatible” as generally used herein means the biological response to the material or implant being appropriate for the implant's intended application in vivo. Any metabolites of these materials should also be biocompatible.

“Blend” as generally used herein means a physical combination of different polymers, as opposed to a copolymer formed of two or more different monomers.

“Compressive modulus” as used herein is measured with a universal testing machine at a cross-head speed of 20 mm min-1. Implants are preloaded to engage the load and compressed at 5 to 15% strain with the load applied along the longitudinal axis of the implant. Clinically relevant cyclic load is repeated 10 times and compressive modulus is calculated based on secondary cyclic load due to the artifact caused by a take up of slack, and alignment or seating of the specimen. Compressive modulus may also be measured using ASTM standards ASTM D1621-16 or ASTM D695-15.

“Copolymers of poly-4-hydroxybutyrate” as generally used herein means any polymer containing 4-hydroxybutyrate with one or more different hydroxy acid units. The copolymers may be isotopically enriched.

“Copolymers of poly(butylene succinate)” as generally used herein means any polymer containing 1,4-butanediol and succinic acid units, and one or more different diol or diacid units or hydroxy acid units. The copolymers may include one or more of the following: branching agent, cross-linking agent, chain extender agent, and reactive blending agent. The copolymers may be isotopically enriched.

“Endotoxin content” as generally used herein refers to the amount of endotoxin present in an implant or sample, and is determined by the limulus amebocyte lysate (LAL) assay.

“Molecular weight” as generally used herein, unless otherwise specified, refers to the weight average molecular weight (Mw), not the number average molecular weight (Mn), and is measured by GPC relative to polystyrene.

“Poly(butylene succinate) mean a polymer containing 1,4-butanediol units and succinic acid units. The polymer may include one or more of the following: branching agent, cross-linking agent, chain extender agent, and reactive blending agent. The polymer may be isotopically enriched.

“Poly(butylene succinate) and copolymers” includes polymers and copolymers prepared with one or more of the following: chain extenders, coupling agents, cross-linking agents and branching agents.

“Poly-4-hydroxybutyrate” as generally used herein means a homopolymer containing 4-hydroxybutyrate units. It can be referred to herein as P4HB or TephaFLEX® biomaterial (manufactured by Tepha, Inc., Lexington, MA). The polymers may be isotopically enriched.

“Soft tissue” as used herein means body tissue that is not hardened or calcified. Soft tissue excludes hard tissues such as bone and tooth enamel.

“Strength retention” refers to the amount of time that a material maintains a particular mechanical property following implantation into a human or animal. For example, if the tensile strength of a resorbable fiber or strut decreases by half over 3 months when implanted into an animal, the fiber or strut's strength retention at 3 months would be 50%.

“Surface roughness” (Ra) as used herein is the arithmetic average of the absolute values of the profile height deviations from a mean line, recorded within an evaluation length.

### II. Materials for Preparing Implants

In embodiments, the implants can be used to form a nipple, reshape a nipple, reconstruct a nipple, modify a nipple, or replace a nipple that has been damaged or surgically removed. The implants can eliminate the need for donor site surgery during nipple reconstruction. The implants are biocompatible, and are preferably replaced in vivo by the patient's tissue as the implants degrade. The implants have a compressive modulus suitable for reconstruction of the nipple. Optionally, the implants can be coated or filled with a hydrogel, bioactive agent, autologous tissue, autologous fat, fat lipoaspirate, injectable fat, adipose cells, fibroblast cells, and stem cells prior to implantation, during implantation, or post-implantation.

### A. Polymers for Preparing Implants

The macroporous network of the implant may comprise permanent materials, such as non-degradable thermoplastic polymers, including polymers and copolymers of ethylene and propylene, including ultra-high molecular weight polyethylene, ultra-high molecular weight polypropylene, nylon, polyesters such as poly(ethylene terephthalate), poly(tetrafluoroethylene), polyurethanes, poly(ether-urethanes), poly(methylmethacrylate), polyether ether ketone, polyolefins, and poly(ethylene oxide). However, the macroporous network of the implant preferably comprises absorbable materials, more preferably thermoplastic or polymeric absorbable materials, and even more preferably the implant and the implant's macroporous network are made completely from absorbable materials.

In a preferred embodiment, the implant's macroporous network is made from one or more absorbable polymers or copolymers, preferably absorbable thermoplastic polymers and copolymers, and even more preferably absorbable thermoplastic polyesters. The implant's macroporous network may, for example, be prepared from polymers including, but not limited to, polymers comprising glycolic acid, glycolide, lactic acid, lactide, 1,4-dioxanone, trimethylene carbonate, 3-hydroxybutyric acid, 4-hydroxybutyrate, 3-hydroxyhexanoate, 3-hydroxyoctanoate, ¿-caprolactone, including polyglycolic acid, polylactic acid, polydioxanone, polycaprolactone, copolymers of glycolic and lactic acids, such as VICRYL® polymer, MAXON® and MONOCRYL® polymers, and including poly(lactide-co-caprolactones); poly(orthoesters); polyanhydrides; poly(phosphazenes); polyhydroxyalkanoates; synthetically or biologically prepared polyesters; polycarbonates; tyrosine polycarbonates; polyamides (including synthetic and natural polyamides, polypeptides, and poly(amino acids)); polyesteramides; poly(alkylene alkylates); polyethers (such as polyethylene glycol, PEG, and polyethylene oxide, PEO); polyvinyl pyrrolidones or PVP; polyurethanes; polyetheresters; polyacetals; polycyanoacrylates; poly(oxyethylene)/poly(oxypropylene) copolymers; polyacetals, polyketals; polyphosphates; (phosphorous-containing) polymers; polyphosphoesters; polyalkylene oxalates; polyalkylene succinates; poly(maleic acids); silk (including recombinant silks and silk derivatives and analogs); chitin; chitosan; modified chitosan; biocompatible polysaccharides; hydrophilic or water soluble polymers, such as polyethylene glycol, (PEG) or polyvinyl pyrrolidone (PVP), with blocks of other biocompatible or biodegradable polymers, for example, poly(lactide), poly(lactide-co-glycolide), or polycaprolactone and copolymers thereof, including random copolymers and block copolymers thereof.

Preferably the macroporous network of the implant is prepared from an absorbable polymer or copolymer that will be substantially absorbed after implantation within a 1 to 24-month timeframe, more preferably a 3 to 18-month timeframe, and retain some residual strength for at least 2 weeks to 6 months.

Blends of polymers and copolymers, preferably absorbable polymers, can also be used to prepare the implant's macroporous network. Particularly preferred blends of absorbable polymers are prepared from absorbable polymers including, but not limited to, polymers comprising glycolic acid, glycolide, lactic acid, lactide, 1,4-dioxanone, trimethylene carbonate, 3-hydroxybutyric acid, 4-hydroxybutyrate, ¿-caprolactone, 1,4-butanediol, 1,3-propane diol, ethylene glycol, glutaric acid, malonic acid, oxalic acid, succinic aid, adipic acid, or copolymers thereof.

In a particularly preferred embodiment, poly-4-hydroxybutyrate (Tepha's P4HB™ polymer, Lexington, MA) or a copolymer thereof is used to make the implant's macroporous network. Copolymers include P4HB with another hydroxy acid, such as 3-hydroxybutyrate, and P4HB with glycolic acid or lactic acid monomer. Poly-4-hydroxybutyrate is a strong, pliable thermoplastic polyester that is biocompatible and resorbable (Williams, et al. Poly-4-hydroxybutyrate (P4HB): a new generation of resorbable medical devices for tissue repair and regeneration, Biomed. Tech. 58(5):439-452 (2013)). Upon implantation, P4HB hydrolyzes to its monomer, and the monomer is metabolized via the Krebs cycle to carbon dioxide and water. In a preferred embodiment, the P4HB homopolymer and copolymers thereof have a weight average molecular weight, Mw, within the range of 50 kDa to 1,200 kDa (by GPC relative to polystyrene), more preferably from 100 kDa to 600 kDa, and even more preferably 200 kDa to 450 kDa. A weight average molecular weight of the polymer of 50 kDa or higher is preferred for processing and mechanical properties.

In another preferred embodiment, the macroporous network of the implant is prepared from a polymer comprising at least a diol and a diacid. In a particularly preferred embodiment, the polymer used to prepare the macroporous network is poly(butylene succinate) (PBS) wherein the diol is 1,4-butanediol and the diacid is succinic acid. The poly(butylene succinate) polymer may be a copolymer with other diols, other diacids or a combination thereof. For example, the polymer may be a poly(butylene succinate) copolymer that further comprises one or more of the following: 1,3-propanediol, ethylene glycol, 1,5-pentanediol, glutaric acid, adipic acid, terephthalic acid, malonic acid, methylsuccinic acid, dimethylsuccinic acid, and oxalic acid. Examples of preferred copolymers are: poly(butylene succinate-co-adipate), poly(butylene succinate-co-terephthalate), poly(butylene succinate-co-butylene methylsuccinate), poly(butylene succinate-co-butylene dimethylsuccinate), poly(butylene succinate-co-ethylene succinate) and poly(butylene succinate-co-propylene succinate). In embodiments, the polymer may be a poly(butylene succinate) copolymer further comprising a hydroxy acid. Examples of hydroxy acids are: glycolic acid and lactic acid. The poly(butylene succinate) polymer or copolymer may also further comprise one or more of the following: chain extender, coupling agent, cross-linking agent and branching agent. For example, poly(butylene succinate) or copolymer thereof may be branched or cross-linked by adding one or more of the following agents: malic acid, trimethylol propane, glycerol, trimesic acid, citric acid, glycerol propoxylate, and tartaric acid. Particularly preferred agents for branching or crosslinking the poly(butylene succinate) polymer or copolymer thereof are hydroxycarboxylic acid units. Preferably the hydroxycarboxylic acid unit has two carboxylic groups and one hydroxyl group, two hydroxyl groups and one carboxyl group, three carboxyl groups and one hydroxyl group, or two hydroxyl groups and two carboxyl groups. In one preferred embodiment, the implant's macroporous network is prepared from poly(butylene succinate) comprising malic acid as a branching or cross-linking agent. This polymer may be referred to as poly(butylene succinate) cross-linked with malic acid, succinic acid-1,4-butanediol-malic acid copolyester, or poly(1,4-butylene glycol-co-succinic acid), cross-linked with malic acid. It should be understood that references to malic acid and other cross-linking agents, coupling agents, branching agents and chain extenders include polymers prepared with these agents wherein the agent has undergone further reaction during processing. For example, the agent may undergo dehydration during polymerization. Thus, poly(butylene succinate)-malic acid copolymer refers to a copolymer prepared from succinic acid, 1,4-butanediol and malic acid. In an embodiment, the poly(butylene succinate)-malic acid copolymer may further comprise one or more hydroxy acids, such as glycolic acid and lactic acid. In another preferred embodiment, malic acid may be used as a branching or cross-linking agent to prepare a copolymer of poly(butylene succinate) with adipate, which may be referred to as poly[(butylene succinate)-co-adipate] cross-linked with malic acid. As used herein, “poly(butylene succinate) and copolymers” includes polymers and copolymers prepared with one or more of the following: chain extenders, coupling agents, cross-linking agents and branching agents. In a particularly preferred embodiment, the poly(butylene succinate) and copolymers thereof contain at least 70%, more preferably 80%, and even more preferably 90% by weight of succinic acid and 1,4-butanediol units. The polymers comprising diacid and diols, including poly(butylene succinate) and copolymers thereof and others described herein, preferably have a weight average molecular weight (Mw) of 10,000 to 400,000, more preferably 50,000 to 300,000 and even more preferably 100,000 to 200,000 based on gel permeation chromatography (GPC) relative to polystyrene standards. In a particularly preferred embodiment, the polymers and copolymers have a weight average molecular weight of 50,000 to 300,000, and more preferably 75,000 to 300,000. In one preferred embodiment, the poly(butylene succinate) or copolymer thereof used to make the macroporous network has one or more, or all of the following properties: density of 1.23-1.26 g/cm³, glass transition temperature of −31° C. to −35° C., melting point of 113° C. to 117° C., melt flow rate (MFR) at 190° C./2.16 kgf of 2 to 10 g/10 min, and tensile strength of 30 to 60 MPa.

In another embodiment, the polymers and copolymers described herein that are used to prepare the macroporous network of the implant, including P4HB and copolymers thereof and PBS and copolymers thereof, include polymers and copolymers in which known isotopes of hydrogen, carbon and/or oxygen are enriched. Hydrogen has three naturally occurring isotopes, which include ¹H (protium), ²H (deuterium) and ³H (tritium), the most common of which is the ¹H isotope. The isotopic content of the polymer or copolymer can be enriched for example, so that the polymer or copolymer contains a higher than natural ratio of a specific isotope or isotopes. The carbon and oxygen content of the polymer or copolymer can also be enriched to contain higher than natural ratios of isotopes of carbon and oxygen, including, but not limited to ¹³C, ¹⁷O or ¹⁸O. Other isotopes of carbon, hydrogen and oxygen are known to one of ordinary skill in the art. A preferred hydrogen isotope enriched in P4HB or copolymer thereof or PBS or copolymer thereof is deuterium, i.e. deuterated P4HB or copolymer thereof or deuterated PBS or copolymer thereof. The percent deuteration can be up to at least 1% and up to 5, 10, 15, 20, 25, 30, 35, 40, 45, 50, 55, 60, 65, 70, 75, 80 or 85% or greater.

In a preferred embodiment, the polymers and copolymers that are used to prepare the macroporous network, including P4HB and copolymers thereof and PBS and copolymers thereof, have low moisture contents. This is preferable to ensure the implants can be produced with high tensile strength, prolonged strength retention, and good shelf life. In a preferred embodiment, the polymers and copolymers that are used to prepare the implants have a moisture content of less than 1,000 ppm (0.1 wt %), less than 500 ppm (0.05 wt %), less than 300 ppm (0.03 wt %), more preferably less than 100 ppm (0.01 wt %), and even more preferably less than 50 ppm (0.005 wt %).

The compositions used to prepare the implants desirably have a low endotoxin content. In preferred embodiments, the endotoxin content is low enough so that the implants produced from the polymer compositions have an endotoxin content of less than 20 endotoxin units per device as determined by the limulus amebocyte lysate (LAL) assay. In one embodiment, the polymeric compositions used to prepare the macroporous network of the implant have an endotoxin content of <2.5 EU/g of polymer or copolymer. For example, the P4HB polymer or copolymer, or PBS polymer or copolymer have an endotoxin content of <2.5 EU/g of polymer or copolymer.

### B. Additives

Certain additives may be incorporated into the implant, preferably in the polymeric compositions that are used to make the macroporous network. In one embodiment, these additives are incorporated with the polymers or copolymers described herein during a compounding process to produce pellets that can be subsequently processed to produce the macroporous networks. If necessary, powders used for processing may be sieved to select an optimum particle size range. In another embodiment, the additives may be incorporated into the polymeric compositions used to prepare the macroporous networks of the implants using a solution-based process.

In a preferred embodiment, the additives are biocompatible, and even more preferably the additives are both biocompatible and absorbable.

In one embodiment, the additives may be nucleating agents and/or plasticizers. These additives may be added to the polymeric compositions used to prepare the macroporous networks of the implants in sufficient quantity to produce the desired result. In general, these additives may be added in amounts between 1% and 20% by weight. Nucleating agents may be incorporated to increase the rate of crystallization of the polymer, copolymer or blend. Such agents may be used, for example, to facilitate fabrication of the macroporous network, and to improve the mechanical properties of the macroporous network. Preferred nucleating agents include, but are not limited to, salts of organic acids such as calcium citrate, polymers or oligomers of PHA polymers and copolymers, high melting polymers such as PGA, talc, micronized mica, calcium carbonate, ammonium chloride, and aromatic amino acids such as tyrosine and phenylalanine.

Plasticizers that may be incorporated into the polymeric compositions for preparing the macroporous networks of the implants include, but are not limited to, di-n-butyl maleate, methyl laureate, dibutyl fumarate, di(2-ethylhexyl) (dioctyl) maleate, paraffin, dodecanol, olive oil, soybean oil, polytetramethylene glycols, methyl oleate, n-propyl oleate, tetrahydrofurfuryl oleate, epoxidized linseed oil, 2-ethyl hexyl epoxytallate, glycerol triacetate, methyl linoleate, dibutyl fumarate, methyl acetyl ricinoleate, acetyl tri(n-butyl) citrate, acetyl triethyl citrate, tri(n-butyl) citrate, triethyl citrate, bis(2-hydroxyethyl) dimerate, butyl ricinoleate, glyceryl tri-(acetyl ricinoleate), methyl ricinoleate, n-butyl acetyl ricinoleate, propylene glycol ricinoleate, diethyl succinate, diisobutyl adipate, dimethyl azelate, di(n-hexyl) azelate, tri-butyl phosphate, and mixtures thereof. Particularly preferred plasticizers are citrate esters.

### C. Bioactive Agents, Cells and Tissues

The implants can be loaded, filled, coated, or otherwise incorporated with bioactive agents. Bioactive agents may be included in the implants for a variety of reasons. For example, bioactive agents may be included in order to improve tissue in-growth into the implant, to improve tissue maturation, to provide for the delivery of an active agent, to improve wettability of the implant, to prevent infection, and to improve cell attachment. The bioactive agents may also be incorporated into the macroporous network of the implant.

The implants can contain active agents designed to stimulate cell in-growth, including growth factors, cell adhesion factors including cell adhesion polypeptides, cellular differentiating factors, cellular recruiting factors, cell receptors, cell-binding factors, cell signaling molecules, such as cytokines, and molecules to promote cell migration, cell division, cell proliferation and extracellular matrix deposition. Such active agents include fibroblast growth factor (FGF), transforming growth factor (TGF), platelet derived growth factor (PDGF), epidermal growth factor (EGF), granulocyte-macrophage colony stimulation factor (GMCSF), vascular endothelial growth factor (VEGF), insulin-like growth factor (IGF), hepatocyte growth factor (HGF), interleukin-1-B (IL-1 B), interleukin-8 (IL-8), and nerve growth factor (NGF), and combinations thereof. As used herein, the term “cell adhesion polypeptides” refers to compounds having at least two amino acids per molecule that are capable of binding cells via cell surface molecules. The cell adhesion polypeptides include any of the proteins of the extracellular matrix which are known to play a role in cell adhesion, including fibronectin, vitronectin, laminin, elastin, fibrinogen, collagen types I, II, and V, as well as synthetic peptides with similar cell adhesion properties. The cell adhesion polypeptides also include peptides derived from any of the aforementioned proteins, including fragments or sequences containing the binding domains.

The implants can incorporate wetting agents designed to improve the wettability of the surfaces of the macroporous networks to allow fluids to be easily adsorbed onto the implant surfaces, and to promote cell attachment and or modify the water contact angle of the implant surface. Examples of wetting agents include polymers of ethylene oxide and propylene oxide, such as polyethylene oxide, polypropylene oxide, or copolymers of these, such as PLURONICS®. Other suitable wetting agents include surfactants, emulsifiers, and proteins such as gelatin.

The implants can contain gels, hydrogels or living hydrogel hybrids to further improve wetting properties and to promote cellular growth throughout the macroporous network structures of the implants. Hydrogel hybrids consist of living cells encapsulated in a biocompatible hydrogel, for example, gelatin, methacrylated gelatin (GelMa), silk gels, and hyaluronic acid (HA) gels.

Other bioactive agents that can be incorporated in the implants include antimicrobial agents, in particular antibiotics, disinfectants, oncological agents, anti-scarring agents, anti-inflammatory agents, anesthetics, small molecule drugs, anti-adhesion agents, inhibitors of cell proliferation, anti-angiogenic factors and pro-angiogenic factors, immunomodulatory agents, and blood clotting agents. The bioactive agents may be proteins such as collagen and antibodies, peptides, polysaccharides such as chitosan, alginate, hyaluronic acid and derivatives thereof, nucleic acid molecules, small molecular weight compounds such as steroids, inorganic materials such as hydroxyapatite and ceramics, or complex mixtures such as platelet rich plasma. Suitable antimicrobial agents include: bacitracin, biguanide, triclosan, gentamicin, minocycline, rifampin, vancomycin, cephalosporins, copper, zinc, silver, and gold. Nucleic acid molecules may include DNA, RNA, siRNA, miRNA, antisense or aptamers.

The implants may also contain allograft material and xenograft materials, including acellular dermal matrix material and small intestinal submucosa (SIS).

In another embodiment, the implants may incorporate systems for the controlled release of the therapeutic or prophylactic agents.

In an embodiment, the implants are coated with autograft, allograft or xenograft tissue and cells prior to implantation, during implantation, or after implantation, or any combination thereof. The autologous tissue and cells are preferably one or more of the following: autologous fat, fat lipoaspirate, fat tissue, injectable fat, adipose tissue, adipose cells, fibroblast cells, and stem cells. As will be evident herein, the macroporous network structures of the implants are designed to create not only the shape of a nipple implant, but also a large surface area that can retain tissue and cells to encourage tissue ingrowth.

### III. Methods for Preparing Implants

A variety of methods can be used to manufacture the implants.

In embodiments, the implant is prepared so that it is able to provide one or more of the following: (i) structural support, (ii) a macroporous network scaffold for tissue ingrowth, (iii) a macroporous network scaffold for delivering cells, tissues, collagen, hyaluronic acid, and bioactive agents, including fat, lipoaspirate, adipose cells, fibroblast cells, and stem cells (iv) a structure that can provide mechanical spacing, (v) a structure that can be coated with cells, tissues, collagen, hyaluronic acid, and bioactive agents, including fat, lipoaspirate, adipose cells, fibroblast cells, and stem cells on the inside of the macroporous network by injection using a needle, and (vi) a structure with a compressive modulus of 0.1 kPa to 10 MPa at 5 to 15% strain, or more preferably 5 to 500 kPa at 5 to 15% strain.

### A. Implant Shapes

In an embodiment, the implants are designed so that when manufactured, they are three-dimensional. In embodiments, the implants are designed to be used for the reconstruction of the nipple of the NAC. In embodiments, the implants are designed to create a nipple with a specific shape, size and projection. In embodiments, the implants are designed to create a nipple that matches the contralateral nipple in terms of shape, projection, size and position.

The implant's shape allows the surgeon to increase tissue volume, reconstruct lost or missing tissue or tissue structures, contour tissues, augment tissues, restore nipple function, repair damaged tissue structures, enhance an existing tissue structure, and alter the projection of the nipple. In a preferred embodiment, the implants are used to reconstruct the nipple following mastectomy. In an embodiment, the implants allow the shape of soft tissue structures to be altered, or sculpted, without the use of permanent implants.

In embodiments, the implant has bullet, flange-cylinder, or top-hat type shape. In other embodiments, the implant does not comprise a hemispherical shape at the second end of the cylindrical shape. In embodiments, the implant has a cylindrical shape, or a cylindrical shape with a flange at one end.

In embodiments, the implant does not comprise a flange component protruding from the circular base on the first end of the cylindrical shape. In embodiments, the flange component is porous. In embodiments, the flange is not porous.

In embodiments, the implant is shell-less. In embodiments, the shell completely surrounds the macroporous network. In embodiments, the shell partially surrounds the macroporous network.

In embodiments, the dimensions of the implant may be shaped and sized to create a nipple that matches the contralateral nipple in terms of shape, projection, and size. Preferably, the implant provides symmetry in the size, shape and position of the reconstructed nipple to match a contralateral nipple.

In embodiments, the nipple implants may be sized or shaped to provide a low, moderate or high projection of the nipple. In embodiments, the height h measured between the first end and second end of the implant is 0.1 to 2 cm, more preferably 0.5 to 1.5 cm, and even more preferably 0.3 to 1 cm. The projection of the nipple may also be controlled by selecting the diameter of the cylindrical shape of the implant. In embodiments, the diameter of the first and second bases of the cylindrical shape of the implant is from 2 to 10 mm, and more preferably 4 to 7 mm.

### B. Construction of the Implants

In embodiments, the nipple implants comprise a load bearing macroporous network with an open pore structure. The macroporous network comprises filaments.

In embodiments, the macroporous networks of the implants have pores with widths or diameters of 75 μm to 10 mm, and more preferably 100 μm to 2 mm. In embodiments, the pore sizes of the macroporous network of the implant are the same. In embodiments, the macroporous network of the implant comprises a mixture of pore sizes.

Preferably, the macroporous networks of the implants have an architecture that provides a large surface area and large void volume suitable to allow the macroporous network to be colonized by cells and invaded by tissue.

In embodiments, the average diameters of the filaments are 50 to 800 μm, more preferably 100 to 600 μm, and even more preferably 150 to 550 μm. In embodiments, the distances between the filaments of the implant are between 50 μm and 1 mm, more preferably 100 μm and 1 mm, and even more preferably 200 μm and 1 mm. The average diameters of the filaments and the distances between the filaments may be selected according to the properties of the implant's macroporous network that are desired, including the compression modulus, the porosity, and the infill density, defined as the ratio of volume occupied by filament material in the implant's macroporous network divided by the total volume of the macroporous network expressed as a percentage. In embodiments, the infill density of the implant's macroporous network is from 1 to 60%, and more preferably from 5 to 25%.

In embodiments, the architecture of the implant's macroporous network preferably provides sufficient porosity to makes it possible to coat the inside of the macroporous network with allograft or xenograft cells, preferably autologous cells, including, but not limited to, autologous fat, fat lipoaspirate, lipo-filling, injectable fat, fibroblast cells, and stem cells. The architecture of the implant's macroporous network is also preferably designed to allow the inner surfaces of the macroporous network to be coated with collagen and or hyaluronic acid or derivative thereof.

In embodiments, the dimensions of the pores of the implant's macroporous network are large enough to allow needles to be inserted into the pores of the macroporous network in order to deliver bioactive agents, cells, fat and other compositions by injection. In embodiments, the architecture of the macroporous network is designed to allow needles with gauges of 12-21 to be inserted into the macroporous network. This property allows the macroporous network to be loaded with cells, collagen, bioactive agents and additives, including fat, using a syringe and without damaging the macroporous network. Preferably, the macroporous networks allow insertion of needles into the open pore structure with outer diameters of 0.5 to 3 mm.

The porosity and shapes of the pores of the implant's macroporous network may be tailored by changing the offset or angle between the filaments in each layer.

In embodiments, the implant's shell may be prepared from a stack of concentric filaments at the periphery of the implant's macroporous network enclosing successive layers of parallel filaments.

In embodiments, the macroporous network of the implant comprises an external shell (e.g., shell 120) or coating. In embodiments, the shell has an outer surface and an inner surface that surrounds an interior volume of said shell. The external shell or coating may partially or fully encase the filaments of the implant's macroporous network. In embodiments, the thickness of the shell or coating is from 10 μm to 5 mm, and more preferably 100 μm to 1 mm. In embodiments, the macroporous network is coated with a polymeric composition.

In embodiments, the shell or coating is permeable to a needle.

In embodiments, the shell comprises a foam with interconnected pores. In embodiments, the shell is an open cell foam, more preferably an open cell foam comprising poly-4-hydroxybutyrate or copolymer thereof or poly(butylene succinate) or copolymer thereof.

In embodiments, the shell comprises collagen, and more preferably type I collagen. In embodiments, the shell comprises collagen, and is 0.1 to 5 mm, or more preferably 0.5 to 3 mm in thickness.

In embodiments, the implant comprises a shell wherein the shell has been heat treated to minimize the roughness of the outer surface of the shell.

In embodiments, the implant is constructed from absorbable mesh and or absorbable dry spun. In embodiments, the core of the nipple implant is formed from an absorbable mesh, and more preferably a macroporous absorbable mesh. In embodiments, the core is formed by rolling up a macroporous mesh to form a cylindrical core of the implant. In embodiments, the macroporous mesh is a monofilament mesh. In embodiments, the monofilament mesh has a Marlex design. In embodiments, the monofilament fibers of the monofilament mesh have suture sizes of 5/0 or 6/0. In embodiments, the monofilament fibers comprise poly-4-hydroxybutyrate. In embodiments, the nipple implant is formed by wrapping a dry spun sheet around the cylindrical core of the macroporous mesh, and fixating it in place. In embodiments, the dry spun sheet is microporous. In embodiments, the dry spun sheet comprises poly-4-hydroxybutyrate or copolymer thereof. In embodiments, the implant further comprises a flange.

In embodiments, the core of the implant is formed from dry spun, and preferably absorbable dry spun. In embodiments, the implant is formed by rolling up a sheet of dry spun to form a cylindrical core of the implant. In embodiments, the dry spun is formed from poly-4-hydroxybutyrate or copolymer thereof. In embodiments, the dry spun cylindrical core is wrapped with an outer layer of macroporous mesh, preferably an absorbable macroporous mesh, and even more preferably an absorbable monofilament knitted mesh. In embodiments, the monofilament fibers of the monofilament mesh have suture sizes of 5/0 or 6/0. In embodiments, the monofilament fibers comprise poly-4-hydroxybutyrate. In embodiments, the implant further comprises a flange.

In embodiments, the implant is formed from a composite of a macroporous absorbable mesh and a dry spun sheet. The composite of two layers is folded to form a cylindrical shape with a flange, wherein the mesh is located in the core of the cylindrical shape and the dry spun is located on the outer surface of the implant. In embodiments, the macroporous mesh is a knitted monofilament mesh. In embodiments, the diameter of the monofilament is a size 5/0 or a size 6/0. In embodiments, the mesh and dry spun sheet are formed from poly-4-hydroxybutyrate or copolymer thereof.

In embodiments, the implant is formed of an exterior body having a base, a hollow cylindrical portion projecting from the base, and at least a partially dome-shape at an end of the hollow cylindrical portion opposite the base. The exterior body defines an internal cavity. An interior load bearing body is located within and least partially fills the internal cavity. In embodiments, each of the exterior body and the interior load bearing body are formed of at least one of a knitted, woven or spun absorbable textile.

In embodiments, the implant is formed from a macroporous absorbable mesh. In embodiments, the implant is formed by folding a mesh into a three-dimensional cone shape (see FIG. 3 and Example 3) and optionally fastening the folded mesh, for example, by heat sealing, stitching or gluing. The origami type folding pattern to make the cone are described at: https://devilsfoodkitchen.com/recipe/101-paper-cones/.

### C. Properties of the Implant

In embodiments, the mechanical properties of the macroporous network and optional shell are designed to provide an implant with an initial compressive modulus that decreases 3-6 months after implantation.

In one embodiment, the compressive modulus of the implant is 0.1 kPa to 10 MPa at 5 to 15% strain, more preferably 1 MPa to 10 MPa at 5 to 15% strain, and even more preferably 1 MPa to 5 MPa at 5 to 15% strain.

In embodiments, the planes of filaments present in the macroporous network of the nipple implant are formed from a polymeric composition. The polymeric composition preferably has one or more of the following properties: (i) an elongation at break greater than 100%; (ii) an elongation at break greater than 200%; (iii) a melting temperature of 60° C. or higher, (iv) a melting temperature higher than 100° C. (v) a glass transition temperature of less than 0° C. (vi) a glass transition temperature between −55° C. and 0° C. (vii) a tensile modulus less than 300 MPa, and (viii) a tensile strength higher than 25 MPa.

In embodiments, the planes of filaments present in the macroporous network of the nipple implant have one or more of the following properties: (i) breaking load of 0.1 to 200 N, 1 to 100 N, or 2 to 50 N; (ii) elongation at break of 10% to 1,000%, more preferably 25% to 500%, and even more preferably greater than 100% or 200%, and (iii) elastic modulus of 0.05 to 1,000 MPa, and more preferably 0.1 to 200 MPa.

In order to allow tissue ingrowth into the macroporous network of the implant, the macroporous network should have a strength retention long enough to permit cells to invade the implant's macroporous network and proliferate. In embodiments, the macroporous network of the implant has a strength retention of at least 25% at 2 weeks, more preferably at least 50% at 2 weeks, and even more preferably at least 50% at 4 weeks. In other embodiments, the macroporous network of the implant is designed to support mechanical forces acting on the implant, and to allow a steady transition of mechanical forces from the macroporous network to regenerated host tissues. In particular, the macroporous network of the implant is designed to support mechanical forces acting on the implant, and to allow a steady transition of mechanical forces to new host tissues.

### D. Other Features of the Implants

The implants or macroporous networks of the implants may be trimmed or cut with scissors, blades, other sharp cutting instruments, or thermal knives in order to provide the desired implant or macroporous network shapes. The implants or macroporous networks can also be cut into the desired shapes using laser-cutting techniques. This can be particularly advantageous in shaping filament-based implants because the technique is versatile, and importantly can provide shaped implants and macroporous networks without sharp edges.

The implants may comprise retainers, such as barbs or tacks, so that the implant can be anchored in the body without the use of sutures. The implants preferably contain the retainers on the circumference of the first circular base of the implant or on the flange. In embodiments, the retainers are preferably located on the implant to allow the implant to be anchored to the breast.

The implant may comprise suture tabs so that the implants can be anchored in the body using for example sutures and or staples. The number of tabs may vary. In embodiments, the implant comprises 1, 2, 3, 4, tabs or more. The tabs attached to the implant must have sufficient strength retention in vivo to resist mechanical loads, and to allow sufficient ingrowth of tissue into the implant in order to prevent subsequent movement of the implant after implantation. In a preferred embodiment, the suture pullout strength of the tabs attached to the implant, is greater than 10 N, and more preferably greater than 20 N.

### E. Implant Coatings and Fillings

The macroporous network of the implant comprises a network wherein there is a continuous path through the network which encourages and allows tissue ingrowth into the implant. The continuous path also allows the entire macroporous network to be coated with one or more of the following: bioactive agents, collagen, hyaluronic acid or derivative thereof, additives, and cells, including fat and fat cells.

In one embodiment, 25% to 100% and more preferably 75% to 100% of the void space of the implant's macroporous network is filled with one or more of the following: cells, collagen, and bioactive agents, including fat, lipoaspirate, adipose cells, fibroblast cells, and stem cells.

The cells and other compositions, such as collagen, hyaluronic acid or derivative thereof, and other bioactive agents, may be coated on the macroporous network prior to implantation, after implantation, or both before and after implantation.

In embodiments, the implants are fabricated with coatings and or some or all of the macroporous network is used as a carrier. For example, the macroporous network may be fabricated by populating some or all of the void space of the macroporous network with one or more of the following: cells, including autograft, allograft and xenograft cells. Examples of cells that can be inserted into the void spaces of the implant's macroporous network, and coated on the surfaces of the macroporous network, include fibroblast cells, and stem cells. In a preferred embodiment, autologous fat, fat lipoaspirate, or injectable fat, is coated on the implant's macroporous network and or inserted into void space of the implant's macroporous network. In yet another embodiment, the implant's macroporous network can be coated or partially or fully filled with one or more bioactive agents. Particularly preferred bioactive agents that can be coated on the implant's macroporous network or used to partially or completely fill the implant's macroporous network include collagen and hyaluronic acid or derivative thereof. In other embodiments, the implant's macroporous network may be coated with one or more antibiotics.

Any suitable method can be used to coat the implant's macroporous network and fill its void space with cells, bioactive agents and other additives. In embodiments, the implant's macroporous network is filled or coated with cells, bioactive agents and other additives by injection, spraying, or dip-coating. Collagen may be applied to the implant's macroporous network by coating and freeze-drying. In a particularly preferred embodiment, the implant's macroporous network may be coated or partially or completely filled with cells, bioactive agents and or other additives by injection using needles that can be inserted into the macroporous network of the implant preferably without damaging the macroporous network. In one embodiment, the needles used for injection of cells, fat, fat lipoaspirate, bioactive agents, collagen, hyaluronic acid or derivative thereof, and other additives have outer diameters between 0.5 mm and 5 mm.

### IV. Methods for Implanting the Implants

In embodiments, the implant is implanted into the body. Preferably, the implant is implanted into a site of reconstruction, remodeling, repair, and or regeneration. In embodiments, the implant is implanted in a patient to form a nipple, reshape a nipple, reconstruct a nipple, modify a nipple, or replace tissue that has been damaged or surgically removed.

In a preferred embodiment, the implant is implanted into a tissue enclosure on the breast mound of a patient. In embodiments, connective tissue and or vasculature will invade the macroporous network of the implant after implantation. In a particularly preferred embodiment, the implant comprises absorbable materials, and connective tissue and or vasculature will also invade the spaces where the absorbable materials have degraded. The pores of the macroporous network may be colonized by cells prior to implantation or, more preferably, following implantation, and the pores of the implant's macroporous network invaded by tissue, blood vessels or a combination thereof.

The implant's macroporous network may be coated or filled with transplantation cells, stem cells, fibroblast cells, adipose cells, and or tissues prior to implantation, or after implantation. In embodiments, the implant's macroporous network is coated or filled with differentiated cells prior to, or subsequent to, implantation. Differentiated cells have a specific form and function. An example is a fat cell. In embodiments, the implant's macroporous network is populated with cells by injection, before or after implantation, and more preferably by using needles that do not damage the macroporous network of the implant. The implant's macroporous network may also be coated or filled with platelets, extracellular adipose matrix proteins, gels, hydrogels, and bioactive agents prior to implantation. In an embodiment, the implant's macroporous network may be coated with antibiotic prior to implantation, for example, by dipping the implant in a solution of antibiotic.

The implants may be used to deliver autologous cells and tissue to the patient. The autologous tissue is preferably one or more of the following: autologous fat, fat lipoaspirate, injectable fat, adipose cells, fibroblast cells, and stem cells.

The implants may be used to deliver fat tissue to a patient. In a particularly preferred embodiment, autologous fatty tissue is prepared prior to, or following, implantation of the implant, and is injected or otherwise inserted into or coated on the implant's macroporous network prior to or following implantation of the implant. The autologous fatty tissue is preferably prepared by liposuction at a donor site on the patient's body. After centrifugation, the lipid phase containing adipocytes is then separated from blood elements, and combined with the implant's macroporous network prior to implantation, or injected, or otherwise inserted into the implant's macroporous network following implantation. In an embodiment, the implant's macroporous network is injected with, or filled with, a volume of lipoaspirate that represents 1% to 50% of the total volume of the macroporous network, and more preferably 1% to 20% of the total volume of the macroporous network.

In another embodiment, lipoaspirate fatty tissue taken from the patient may be mixed with a biological or synthetic matrix, such as very small fibers or particles, prior to adding the lipoaspirate to the implant's macroporous network. In this embodiment, the added matrix serves to hold or bind micro-globules of fat, and disperse and retain them within the macroporous network of the implant.

In an embodiment, an implant is implanted on the tissue mound of a breast. In an embodiment, implants are implanted on the tissue mounds of both breasts of a patient.

In a particularly preferred embodiment, the implant is implanted in a patient that has undergone a mastectomy.

In an embodiment, the implant is inserted in a tissue enclosure formed at the site of nipple reconstruction.

In a preferred embodiment, the implant is implanted by a method comprising: making an incision in a patient to create a tissue enclosure that is configured to receive a nipple implant; and inserting the nipple implant into the tissue enclosure, wherein the tissue enclosure is configured to conform around the nipple implant. In embodiments, the method of implanting the implant comprises configuring an incision to create tissue flaps with opposable edges, such that when the edges are brought together the tissue flaps form a void for receiving the nipple implant so that the inner surface of the tissue flaps are in contact with the nipple implant. In embodiments, the method of implanting the implant comprises making an incision with a CV-flap incision path, a S-flap incision path, or a star-flap incision path.

In an embodiment, the implant is implanted by a method comprising: (i) making one or more incisions on the breast mound of a reconstructed patient breast to create free-moving skin flaps, (ii) manipulating and fixating the skin flaps to produce a projecting tissue enclosure, (iii) inserting a nipple implant into the tissue enclosure, (iv) opposing the patient's tissue against the external surface of the nipple implant, and (v) fixating the tissue enclosure to enclose the implant within the tissue enclosure. In an embodiment, the method further comprises suturing the skin flaps to form a projecting tissue enclosure. In an embodiment, the method further comprises suturing the tissue enclosure to enclose the implant within the tissue enclosure. In embodiments, the tissue enclosure is sized so that there is little if any dead space between the implant and patient tissue. In embodiments, the tissue enclosure is sized to conform to the volume of the implant.

In embodiments, the method of implantation comprises implanting the first end of the cylindrical shape of the implant posterior to the second end of the cylindrical shape of the implant. In a particularly preferred embodiment, the method of implantation comprises implanting the first end of the cylindrical shape of the implant posterior to the second end of the of the implant. In embodiments, the hemispherical shape of the implant is implanted beneath the skin of the patient, and the first end of the cylindrical shape of the implant is implanted on the breast mound of the patient.

In embodiments, the implant comprises a flange on the first end of the cylindrical shape of the implant, and the method of implantation comprises implanting the flange of the implant on the breast mound and posterior to the second end of the cylindrical shape of the implant.

The implant's macroporous network may be coated or filled with cells and tissues prior to, or subsequent to, implantation, as well as with cytokines, platelets and extracellular adipose matrix proteins. The implant's macroporous network may also be coated or filled with other tissue cells, such as stem cells genetically altered to contain genes for treatment of patient illnesses.

In an embodiment, the implant has properties that allows it to be delivered by minimally invasive means through a small incision. The implant may, for example, be designed so that it can be rolled, folded or compressed to allow delivery through a small incision. In an embodiment, the implant has a three-dimensional shape and shape memory properties that allow it to assume its original three-dimensional shape unaided after it has been delivered through an incision and into a tissue enclosure. For example, the implant may be temporarily deformed by rolling it up into a small diameter cylindrical shape, delivered using an inserter, and then allowed to resume its original three-dimensional shape unaided in vivo.

### EXAMPLES

Embodiments of the present invention will be further understood by reference to the following non-limiting examples.

### Example 1: Nipple Implant with a Core of Macroporous P4HB Monofilament Knitted Mesh and a P4HB Microporous Dry Spun Shell

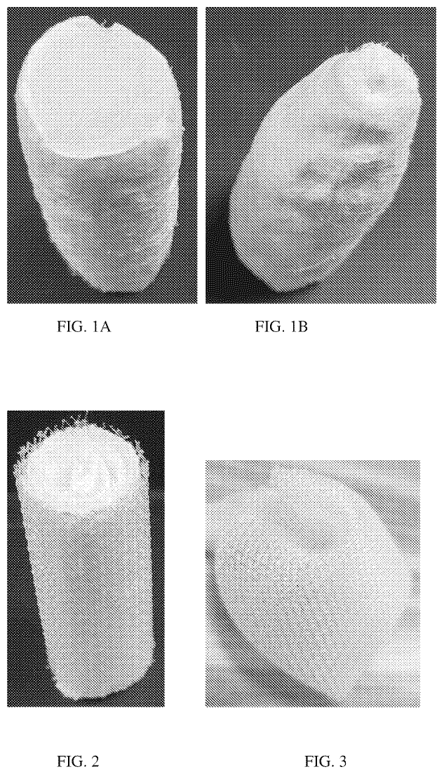

A macroporous poly-4-hydroxybutyrate (P4HB) knitted mesh with a Marlex design was prepared from P4HB monofilament fiber (size 5/0), cut to size, and rolled up to form the core of a nipple implant. A microporous P4HB dry spun (P4HB Mw 250-400 kDa) sheet was prepared by solution spinning an 8% w/v solution of P4HB in chloroform through a 1.0 mm annular spinneret (1.1 mm inner diameter and 2.1 mm outer diameter) using dried compressed air (3 bar). The dry spun sheet had a thickness of 162 μm, a density of 4.5 mg/cm², and an average fiber diameter of 3.9±4.3 μm. The dry spun sheet was cut into a rectangular shape measuring 12 mm×9.5 mm, and rolled around the core of macroporous P4HB monofilament. The free edge of the rolled meshes, including the top portion of the implant, was heat-sealed at 80° C. for 3 seconds to fix it in place. The same heat scaling was performed to fixate the dry spun shell around the mesh core and form the implant shown in FIGS. 1A and 1B.

### Example 2: Nipple Implant Formed from a Composite Structure of P4HB Monofilament Knitted Mesh and P4HB Dry Spun

A macroporous poly-4-hydroxybutyate (P4HB) knitted mesh with a Marlex design was prepared from P4HB monofilament fiber (size 5/0), and cut to size. A P4HB (Mw 250-400 kDa) dry spun sheet was prepared by solution spinning an 8% w/v solution of P4HB in chloroform through a 1.0 mm annular spinneret (1.1 mm inner diameter and 2.1 mm outer diameter) using dried compressed air (3 bar), and cut to the same size as the mesh. The dry spun sheet was overlaid on the mesh to form a composite, and stitched along one edge using P4HB fiber (suture size 5/0). A cylindrical shaped implant was formed by flipping the stitched edge of the composite, and rolling up the composite (like a Swiss roll). The edge of the composite was cut, and fixated with fibrin glue forming a cylinder with alternating layers of monofilament mesh and dry spun as shown in FIG. 2.

### Example 3: Cone-Shaped Mesh Nipple Implant

A macroporous scaffold was made of poly-4-hydroxybutyrate (P4HB) extruded monofilament (0.165 mm, MW 285 kDa) knitted using a 14-gauge double needle bar machine with Marlex pattern. The P4HB mesh density was 150 g/m² approximately.

A nipple implant, depicted in FIG. 3, was prepared from a macroporous P4HB scaffold by folding a two-dimensional mesh into a three-dimensional cone shape. The origami type folding pattern to make the cone is described at: https:/devilsfoodkitchen.com/recipe/101-paper-cones/. The nipple implant shown in FIG. 3 was formed by cutting a rectangular two-dimensional P4HB mesh in half diagonally from one corner to the other to form two triangular pieces of P4HB mesh. The short side of one triangular piece was folded to align with the altitude intersecting the longest side of the triangle thereby forming a cone. The next longest side of the triangular piece was wrapped around the cone, and the tail folded down inside the cone shape to lock the cone shape of the implant. The cone shape may optionally be fastened, for example, by heat sealing, stitching or gluing.

### Example 4: Thermo-Shaped Mesh Nipple Implant

A macroporous scaffold was made of poly-4-hydroxybutyrate (P4HB) extruded monofilament (0.165 mm, MW 285 kDa) knitted using a 14-gauge double needle bar machine with Marlex pattern. The P4HB mesh density was 150 g/m² approximately.

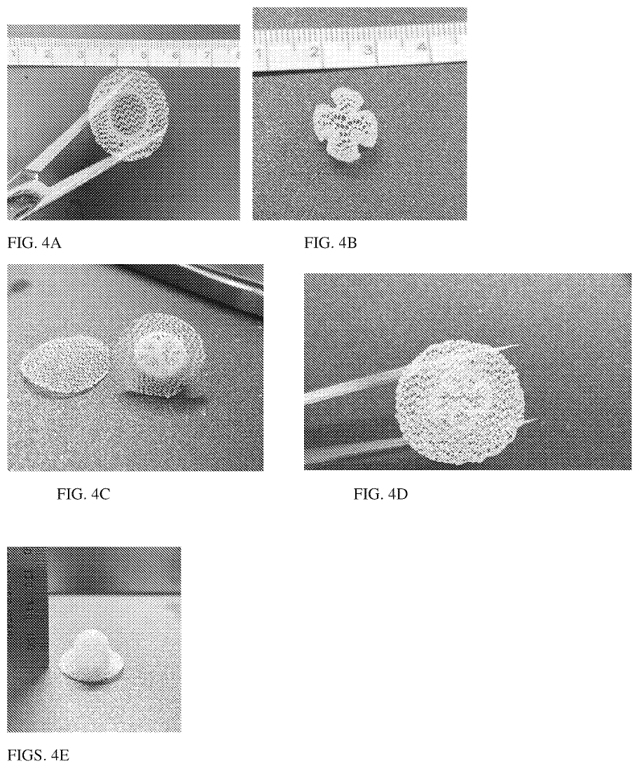

A nipple implant, shown in FIGS. 4A-4E, was prepared from a macroporous P4HB scaffold. An exterior body of the implant has an annular base, a substantially cylindrical portion extending upwardly from the annular base, and a dome-shape above the cylindrical portion. An interior body is located within an internal cavity defined by the exterior body. To form the nipple implant, P4HB mesh was cut into two circular sheets of 5 cm radius using a laser cutter. A mesh piece was placed into a positive cylindrical mold and then a vacuum pressure applied. Then, heated fluid (air) was transferred to the system for about 20 seconds to form the thermo-shaped exterior mesh body with an annular base, a cylindrical portion open at one end, and a dome shape at the cylindrical portion opposite the open end (see FIG. 4A). An internal body of the mesh nipple implant was prepared from a second piece of the pre-cut mesh which was placed into a positive conical mold with four (4) grooves at 90 degrees to form (4) folded leaflets. The latter was then placed into a hollow cavity negative mold to secure the mesh within the four grooves, and immersed in a heated water bath at 57° C. for 5 minutes to form the thermo-shaped internal body with folded leaflets (FIG. 4B). The thermo-shaped internal body was inserted through the open end and into the cavity of the thermo-shaped exterior body to provide loading support (see FIG. 4C). A flange (or base) was prepared from another circular sheet of pre-cut P4HB knitted mesh. The flange and the annular base of the thermo-shaped exterior mesh body were then heat sealed to form the mesh-nipple implant (see FIGS. 4D and 4E).

### Example 5: Thermo-Shaped Lightweight Mesh Nipple Implant

The method described in Example 4 was used to prepare a nipple implant, except that the flange was thermo-formed with the internal body before insertion of the internal body through the open end of the cylindrical portion and into the thermo-shaped exterior body.

### Example 6: Mesh Nipple Implant with Loosely Rolled Inner Body

A macroporous scaffold was made of poly-4-hydroxybutyrate (P4HB) extruded monofilament (0.165 mm, MW 285 kDa) knitted using a 14-gauge double needle bar machine with Marlex pattern. The P4HB mesh density was 150 g/m² approximately.

A nipple implant was prepared from a macroporous P4HB scaffold. The implant included an inner body, shown to the left of FIG. 5 and exterior shell shown to the right of FIG. 5. P4HB mesh was cut into a trapezium shape and into a circle shape using a laser cutter. The internal body of the mesh nipple implant was prepared from a pre-cut trapezium mesh rolled over a pin to create a minimum of 5 layers or windings. The 5-layered construct was placed into a hollow cavity mold, the mold placed into a heated water bath at 57° C. for 5 minutes, and then the rolled mesh construct was removed from the mold. The layers created by this technique were equally spaced. The molded interior body was joined at one end to a mesh base. The exterior body of the mesh nipple implant was prepared from a piece of the pre-cut mesh placed into a positive cylindrical mold and then a vacuum pressure applied, forming an annular base with a projecting cylindrical portion and a dome-shaped top. Heated fluid (air) was transferred to the mold for about 20 seconds to form the exterior body. The thermo-shaped internal body was placed into the cavity of the thermo-shaped external body to provide loading support. The annular flange of the exterior body and the base of the internal body were then heat sealed to form the mesh-nipple implant. Alternatively, the internal body could be used without the exterior body.

### Example 7: Mesh Nipple Implant with Tightly Rolled Inner Body

A macroporous scaffold was made of poly-4-hydroxybutyrate (P4HB) extruded monofilament (0.165 mm, MW 285 kDa) knitted using a 14-gauge double needle bar machine with Marlex pattern. The P4HB mesh density was 150 g/m² approximately.

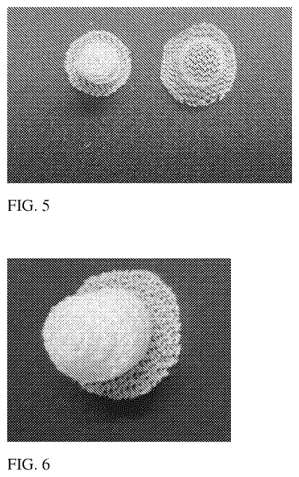

A nipple implant, depicted in FIG. 6, was prepared from a macroporous P4HB scaffold by thermoforming P4HB knitted mesh. Two (2) trapezium shapes and a circle shape were cut from a larger P4HB sheet using a laser cutter. A rolled mesh construct was prepared by rolling the stacked trapezium sheets over a pin to form tight windings. The rolled mesh construct was then placed into a hollow cavity mold. The mold was then placed into a heated water bath at 57° C. for 5 minutes to create a molded mesh roll. A flange was created from the circular mesh shape to form the base of the implant, and the flange heat sealed to the rolled mesh. The implant could be used in this particular form or could be associated with an exterior body as in the preceding examples.

Examples 4-7 could also have been formed with dry spun P4HB, with a composite of dry spun P4HB and knitted P4HB, or with dry spun P4HB wrapped around some or all of the P4HB knitted portions of the nipple implants.

We claim:

1. A nipple implant, comprising:
a first portion of a polymeric knitted or woven macroporous textile and a second portion of polymeric microporous non-woven or foam,
wherein the first portion and the second portion are configured to form a cylindrical body portion and at least a partially dome-shape at an end of the cylindrical body portion.

2. The nipple implant of claim 1 wherein one of the first portion and the second portion is a core of the implant and the other of the first portion and the second portion surrounds the core.

3. The nipple implant of claim 1, further including a flange base at an end of the cylindrical body portion opposite of the at least partially dome-shape end.

4. The nipple implant of claim 2, wherein the first portion includes at least one sheet of microporous poly-4-hydroxybutyrate or copolymer thereof.

5. The nipple implant of claim 2, wherein the first portion includes at least one sheet of spun poly-4-hydroxybutyrate or copolymer thereof.

6. The nipple implant of claim 4, wherein the at least one sheet has been manipulated into a cylindrical shape and an at least partially dome-shape.

7. The nipple implant of claim 2, wherein the second portion includes at least one sheet of spun poly-4-hydroxybutyrate or copolymer thereof.

8. The nipple implant of claim 7, wherein the at least one spun sheet has been manipulated into a cylindrical shape and at least a partially dome-shape.

9. The nipple implant of claim 1, wherein at least one of the first portion and the second portion is at least partly filled with a hydrogel.

10. The nipple implant of claim 1, wherein the first portion comprises a plurality of macropores, and the macropores have an average diameter or average width of 75 to 2,000 microns.

11. The nipple implant of claim 1, wherein filaments included in the first portion have one or more of the following properties: an average diameter of 10 μm to 5 mm, a breaking load of 0.1 to 200 N, an elongation at break of 10 to 1,000%, and an elastic modulus of 0.05 to 1,000 MPa.

12. The nipple implant of claim 11, wherein the filaments included in the first portion have one or more of the following properties: (i) an elongation at break greater than 100%; (ii) an elongation at break greater than 200%; (iii) a melting temperature of 60° C. or higher, (iv) a melting temperature higher than 100° C. (v) a glass transition temperature of less than 0° C. (vi) a glass transition temperature between −55° C. and 0° C., (vii) a tensile modulus less than 300 MPa, and (viii) a tensile strength higher than 25 MPa.

13. The nipple implant of claim 1, wherein the first portion and the second portion are respectively formed from an absorbable polymer comprising, or prepared from, one or more monomers selected from the group: glycolide, lactide, glycolic acid, lactic acid, 1,4-dioxanone, trimethylene carbonate, 3-hydroxybutyric acid, 3-hydroxybutyrate, 3-hydroxyhexanoate, 4-hydroxybutyric acid, 4-hydroxybutyrate, 3-hydroxyoctanoate, ε-caprolactone, 1,4-butanediol, 1,3-propane diol, ethylene glycol, glutaric acid, malic acid, malonic acid, oxalic acid, succinic aid, or adipic acid, or the absorbable polymer comprises poly-4-hydroxybutyrate or copolymer thereof, or poly(butylene succinate) or copolymer thereof.

14. The nipple implant of claim 13, wherein the first portion is formed of poly-4-hydroxybutyrate or copolymer thereof, and wherein the second portion is formed of poly-4-hydroxybutyrate or copolymer thereof.

15. A nipple implant, comprising:
an exterior body having a base, a hollow cylindrical portion projecting from the base, and at least a partially dome-shape at an end of the hollow cylindrical portion opposite the base, the exterior body defining an internal cavity; and
an interior load bearing body located within and at least partially filling the internal cavity;
wherein each of the exterior body and the interior load bearing body are formed of at least one of a knitted, woven or non-woven absorbable textile.

16. The nipple implant of claim 15, wherein the absorbable textile is formed from an absorbable polymer comprising, or prepared from, one or more monomers selected from the group: glycolide, lactide, glycolic acid, lactic acid, 1,4-dioxanone, trimethylene carbonate, 3-hydroxybutyric acid, 3-hydroxybutyrate, 3-hydroxyhexanoate, 4-hydroxybutyric acid, 4-hydroxybutyrate, 3-hydroxyoctanoate, ¿-caprolactone, 1,4-butanediol, 1,3-propane diol, ethylene glycol, glutaric acid, malic acid, malonic acid, oxalic acid, succinic aid, or adipic acid, or the absorbable polymer comprises poly-4-hydroxybutyrate or copolymer thereof, or poly(butylene succinate) or copolymer thereof.

17. The nipple implant of claim 15, wherein the interior load bearing body includes a base and a resilient structure projecting upwardly from the base, the resilient structure occupying only a portion of the internal cavity when viewed in a radial direction.

18. The nipple implant of claim 17, wherein the resilient structure includes at least two adjacent windings, wherein there is a gap when viewed in the radial direction between the two adjacent windings.

19. The nipple implant of claim 17, wherein the resilient structure has a polygon shape.

20. The nipple implant of claim 19, wherein the polygon shape is different from a shape of the internal cavity.

21. The nipple implant of claim 19, wherein the resilient structure of polygon shape is located in the hollow cylindrical portion and the at least partially dome shape of the exterior body.

22. The nipple implant of claim 19, wherein the resilient structure has a clover-leaf shape.

23. The nipple implant of claim 17, wherein the resilient structure has a corrugated surface.
